(19) United States
(12) Patent Application Publication
(10) Pub. No.: US 2026/0139038 A1
(43) Pub. Date: May 21, 2026

(54) METHODS AND COMPOSITIONS FOR DETECTING AND TREATING MYCOPLASMA PNEUMONIAE

(71) Applicant: Board of Regents, The University of Texas System, Austin, TX (US)

(72) Inventors: Thirumalai R. Kannan, San Antonio, TX (US); Joel Baseman, San Antonio, TX (US)

(21) Appl. No.: 19/393,327

(22) Filed: Nov. 18, 2025

Related U.S. Application Data
(60) Provisional application No. 63/721,627, filed on Nov. 18, 2024.

(51) Int. Cl. C07K 16/12; G01N 33/569

(52) U.S. Cl. CPC C07K 16/1253; G01N 33/56933; G01N 2333/30

(57) ABSTRACT

Provided are monoclonal antibodies and antigen-binding fragments that specifically bind the CARDS toxin or the P1 adhesin of Mycoplasma pneumoniae. The antibodies enable rapid, sensitive detection of M. pneumoniae infection and provide therapeutic neutralization of toxin activity or inhibition of bacterial adherence. Also provided are nucleic acids, vectors, host cells, pharmaceutical compositions, diagnostic methods, and kits using these antibodies.

### PRIORITY

This application claims priority to U.S. Provisional Patent Ser. No. 63/721,627 filed Nov. 18, 2024, which is incorporated herein by reference in its entirety.

### STATEMENT REGARDING FEDERALLY FUNDED RESEARCH

This invention was made with government support under U19AI070412 awarded by the National Institutes of Health. The government has certain rights in the invention.

### REFERENCE TO SEQUENCE LISTING

A sequence listing is being submitted electronically with this application. The sequence listing is incorporated herein by reference. The sequence listing that is contained in the file named “UTSK-P0561US” the file is 66,714 bytes (as measured in Microsoft Windows®) and was created on Nov. 18, 2025.

### FIELD

The invention relates generally to biomedicine and, more particularly, to monoclonal antibodies, compositions, and methods for the detection and treatment of Mycoplasma pneumoniae infections.

### BACKGROUND

Mycoplasma pneumoniae is a major cause of community-acquired respiratory tract infections worldwide, particularly in children and young adults. Clinical manifestations range from mild upper respiratory illness to tracheobronchitis and pneumonia. Outbreaks occur frequently in closed populations, and extrapulmonary complications can include neurological, cardiac, and dermatological sequelae. Unlike most bacterial pathogens, M. pneumoniae lacks a cell wall, rendering it intrinsically resistant to β-lactam antibiotics. Macrolides have been the first-line treatment, but macrolide resistance now exceeds 50-90% in some regions, resulting in prolonged illness and increased hospitalization rates. Two M. pneumoniae proteins are recognized as critical virulence determinants: (1) the Community-Acquired Respiratory Distress Syndrome (CARDS) toxin, an ADP-ribosylating protein that induces intense vacuolization, ciliostasis, and inflammatory cytokine release in respiratory epithelium; and (2) the P1 cytadhesin, a surface protein essential for attachment to sialylated oligosaccharides on host respiratory epithelial cells.

Current diagnostic methods rely primarily on PCR or serology. PCR is sensitive but requires specialized equipment and is not widely available as a point-of-care test. Serological assays suffer from delayed seroconversion and cross-reactivity. Rapid, antigen-based detection methods with high specificity remain an unmet need.

There remains a need for additional compositions and methods for detecting and treating Mycoplasma pneumoniae infections. The present disclosure addresses these needs by providing novel monoclonal antibodies that specifically bind CARDS toxin or the P1 adhesin of Mycoplasma pneumoniae.

### SUMMARY

The present disclosure provides novel monoclonal antibodies and antigen-binding fragments thereof that specifically bind either the CARDS toxin or the P1 adhesin of Mycoplasma pneumoniae. These antibodies enable both highly specific diagnostic assays and therapeutic interventions (toxin neutralization and inhibition of bacterial adherence) that are effective even against macrolide-resistant strains.

In one aspect, provided are isolated antibodies or antigen-binding fragments comprising the following CDR combinations (Kabat numbering):
(a) LC-CDR1-3 of SEQ ID NOs: 27-29 and HC-CDR1-3 of SEQ ID NOs: 30-32 (mAb 7F10) (b) LC-CDR1-3 of SEQ ID NOs: 33-35 and HC-CDR1-3 of SEQ ID NOs: 36-38 (mAb 3A12) (c) LC-CDR1-3 of SEQ ID NOs: 39-41 and HC-CDR1-3 of SEQ ID NOs: 42-44 (mAb 5B8) (d) LC-CDR1-3 of SEQ ID NOs: 45-47 and HC-CDR1-3 of SEQ ID NOs: 48-50 (mAb 8E8) (e) LC-CDR1-3 of SEQ ID NOs: 51-53 and HC-CDR1-3 of SEQ ID NOs: 54-56 (mAb 6E7) (f) LC-CDR1-3 of SEQ ID NOs: 57-59 and HC-CDR1-3 of SEQ ID NOs: 60-62 (mAb 21B8)

Further provided are antibodies or fragments having variable light chain and variable heavy chain sequences at least 90%, 95%, 98%, or 99% identical to the pairs SEQ ID NOs: 2/4, 6/8, 10/12, 14/16, 18/20, and 22/24.

Additional embodiments include nucleic acid molecules encoding the antibodies or fragments, recombinant vectors, host cells, pharmaceutical compositions, methods of treatment, methods of detection, and diagnostic kits comprising the disclosed antibodies.

Definitions—The definitions provided herein are to facilitate understanding of certain terms used frequently in this specification and are not intended to limit the scope of the invention beyond the ordinary meaning of the terms as understood by those skilled in the art.

“Antibody” encompasses monoclonal antibodies (including full-length antibodies), polyclonal antibodies, multispecific antibodies (e.g., bispecific antibodies) formed from at least two intact antibodies, and antibody fragments so long as they exhibit the desired antigen-binding activity. Antibody also includes antibodies of any class or subclass (e.g., IgG, IgA, IgM, IgE, IgD).

“Antigen-binding fragment” or “fragment” refers to a portion of an intact antibody that retains specific binding to the same antigen (e.g., CARDS toxin or P1 adhesin). Non-limiting examples include Fab, Fab′, F(ab′)₂, Fv, scFv, dsFv, diabodies, triabodies, tetrabodies, linear antibodies, single-chain antibody molecules, and multispecific antibodies formed from antibody fragments.

“Variable region” or “variable domain” refers to the portion of an antibody light or heavy chain involved in antigen binding. The variable regions of the heavy chain and light chain each typically comprise four framework regions (FRs) and three complementarity-determining regions (CDRs).

“Complementarity-determining region” or “CDR” refers to the hypervariable regions in the light-chain and heavy-chain variable domains as defined by the Kabat numbering system (Kabat et al., Sequences of Proteins of Immunological Interest, 5th Ed. Public Health Service, National Institutes of Health, Bethesda, MD. (1991)).

“Specifically binds” or “binds specifically” means that the antibody or fragment binds Mycoplasma pneumoniae CARDS toxin or P1 adhesin with a K_D of ≤10⁻⁷ M, ≤10⁻⁸ M, ≤10⁻⁹ M, or better as measured by surface plasmon resonance or equivalent method, and exhibits no significant binding to unrelated proteins.

“Neutralizes” or “neutralizing” with respect to CARDS toxin means the antibody or fragment reduces or abolishes ADP-ribosyltransferase activity and/or vacuolating/cytotoxic activity of CARDS toxin by at least 50%, preferably ≥80%, in a suitable in vitro assay.

“Inhibits adherence” or “blocks adherence” with respect to P1 adhesin means the antibody or fragment reduces attachment of M. pneumoniae to respiratory epithelial cells by at least 50%, preferably ≥80%, in a suitable in vitro adherence assay.

“Humanized antibody” refers to a chimeric antibody comprising amino acid residues from non-human CDRs and human framework regions.

“Isolated” (when applied to an antibody, nucleic acid, etc.) means altered or removed from the natural state by the hand of man.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a composition and/or method that “comprises” a list of elements (e.g., components or features or steps) is not necessarily limited to only those elements (or components or features or steps) but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of that embodiment and not intended to imply that the scope of the disclosure, including the claims, is limited to that embodiment.

I. Antibodies and Antibody Fragments that Bind CARDS Toxin or Mycoplasma pneumoniae Major Adhesin Protein P1

The present disclosure provides six monoclonal antibodies that specifically bind Mycoplasma pneumoniae antigens: three directed against the Community-Acquired Respiratory Distress Syndrome (CARDS) toxin (7F10, 3A12, and 5B8) and three directed against the major cytadhesin protein P1 (8E8, 6E7, and 21B8). The amino acid sequences of the target proteins and the complementarity-determining regions (CDRs) of the light and heavy chains of each antibody directed to the target proteins are set forth below:

Full length CARDS toxin acids 1-591 (amino MPNPVRFVYRVDLRSPEEIFEHGFSTLGDVRNFFEHILSTNFGRSYFISTSETPTAAIRFFG SWLREYVPEHPRRAYLYEIRADQHFYNARATGENLLDLMRQRQVVFDSGDREMAQMG IRALRTSFAYQREWFTDGPIAAANVRSAWLVDAVPVEPGHAHHPAGRVVETTRINEPE MHNPHYQELQTQANDQPWLPTPGIATPVHLSIPQAASVADVSEGTSASLSFACPDWSPP SSNGENPLDKCIAEKIDNYNLQSLPQYASSVKELEDTPVYLRGIKTQKTFMLQADPQNN NVFLVEVNPKQKSSFPQTIFFWDVYQRICLKDLTGAQISLSLTAFTTQYAGQLKVHLSVS AVNAVNQKWKMTPQDIAITQFR VSSELLGQTENGLFWNTKSGGSQHDLYVCPLKNPPS DLEELQIIVDECTTHAQFVTMRAASTFFVDVQLGWYWRGYYYTPQLSGWSYQMKTPD GQIFYDLKTSKIFFVQDNQNVFFLHNKLNKQTGYSWDWVEWLKHDMNEDKDENFKW YFSRDDLTIPSVEGLNFRHIRCYADNQQLKVIISGSRWGGWYSTYDK VESNVEDKILVK DGFDRF (SEQ ID NO:25)); N-terminus CARD toxin (N-CARDS; encoding amino acids 1-249 of SEQ ID NO:25) and C-terminus CARDS (C-CARDS; encoding amino acids 273-591 of SEQ ID NO: 25). All the above amino acids encoding DNA fragments were cloned under the NdeI and BamHI site of T7 promoter of pET19b plasmid (Novagen).

Mycoplasma pneumoniae major adhesin protein P1 having amino acids MHQTKKTALSKSTWILILTATASLATGLTVVGHFTSTTTTLKRQQFSYTRPDEVALRHTN AINPRLTPWTYRNTSFSSLPLTGENPGAWALVRDNSAKGITAGSGSQQTTYDPTRTEAA LTASTTFALRRYDLAGRALYDLDFSKLNPQTPTRDQTGQITFNPFGGFGLSGAAPQQWN EVKNKVPVEVAQDPSNPYRFAVLLVPRSVVYYEQLQRGLGLPQQRTESGQNTSTTGAM FGLKVKNAEADTAKSNEKLQGAEATGSSTTSGSGQSTQRGGSSGDTKVKALKIEVKKK SDSEDNGQLQLEKNDLANAPIKRSEESGQSVQLKADDFGTALSSSGSGGNSNPGSPTPW RPWLATEQIHKDLPKWSASILILYDAPYARNRTAIDRVDHLDPKAMTANYPPSWRTPK WNHHGLWDWKARDVLLQTTGFFNPRRHPEWFDGGQTVADNEKTGFDVDNSENTKQG FQKEADSDKSAPIALPFEAYFANIGNLTWFGQALL VFGGNGHVTKSAHTAPLSIGVFRV RYNATGTSATVTGWPYALLFSGMVNKQTDGLKDLPFNNNRWFEYVPRMAVAGAKFV GRELVLAGTITMGDTATVPRLLYDELESNLNLVAQGQGLLREDLQLFTPYGWANRPDL PIGAWSSSSSSSHNAPYYFHNNPDWQDRPIQNVVDAFIKPWEDKNGKDDAKYIYPYRYS GMWAWQVYNWSNKLTDQPLSADFVNENAYQPNSLFAAILNPELLAALPDKVKYGKEN EFAANEYERFNQKLTVAPTQGTNWSHFSPTLSRFSTGFNLVGSVLDQVLDYVPWIGNGY RYGNNHRGVDDITAPQTSAGSSSGISTNTSGSRSFLPTFSNIGVGLKANVQATLGGSQTM ITGGSPRRTLDQANLQLWTGAGWRNDKASSGQSDENHTKFTSATGMDQQGQSGTSAG NPDSLKQDNISKSGDSLTTQDGNAIDQQEATNYTNLPPNLTPTADWPNALSFTNKNNAQ RAQLFLRGLLGSIPVLVNRSGSDSNKFQATDQKWSYTDLHSDQTKLNLPAYGEVNGLL NPALVETYFGNTRAGGSGSNTTSSPGIGFKIPEQNNDSKATLITPGLAWTPQDVGNLVVS GTTVSFQLGGWLVTFTDFVKPRAGYLGLQLTGLDASDATQRALIWAPRPWAAFRGSW VNRLGRVESVWDLKGVWADQAQSDSQGSTTTATRNALPEHPNALAFQVSVVEASAYK PNTSSGQTQSTNSSPYLHLVKPKKVTQSDKLDDDLKNLLDPNQVRTKLRQSFGTDHSTQ PQPQSLKTTTPVFGTSSGNLSSVLSGGGAGGGSSGSGQSGVDLSPVEK VSGWLVGQLPS TSDGNTSSTNNLAPNTNTGNDVVGVGRLSESNAAKMNDDVDGIVRTPLAELLDGEGQT ADTGPQSVKFKSPDQIDFNRLFTHPVTDLFDPVTMLVYDQYIPLFIDIPASVNPKMVRLK VLSFDTNEQSLGLRLEFFKPDQDTQPNNNVQVNPNNGDFLPLLTASSQGPQTLFSPFNQ WPDYVLPLAITVPIVVIVLSVTLGLAIGIPMHKNKQALKAGFALSNQKVDVLTKAVGSV FKEIINRTGISQAPKRLKQTSAAKPGAPRPPVPPKPGAPKPPVQPPKKPA (SEQ ID NO:26) can be used as an antigen. In some aspects, a C-terminal region can also be used as an antigen.

Anti mouse monoclonal antibodies have been generated by producing hybridomas against specific domains of CARDS toxin (recognizing amino acids 260-; amino acids 570-591) and P1 protein. Mouse hybridoma cell lines that produce antibodies against specific epitopes of CARDS toxin: A first hybridoma reacts against an epitope between amino acids 250-273 of SEQ ID NO: 25. A second hybridoma reacts against an epitope between amino acids 570-591 of SEQ ID NO: 25.

A polynucleotide encoding the 7F10 light chain includes the nucleic acid sequence ATGATGAGTCCTGCCCAGTTCCTGTTTCTGTTAGTGCTCTGGATTCGGGAAACCAAC GGTGATGTTGTGCTGACCCAGACTCCACTCACTTTGTCGGTTACCATTGGACAACCA GCCTCCATCTCTTGCAAGTCAAGTCAGAGCCTCTTAGATAGTGATGGAAAGACATAT TTGAATTGGTTGTTACAGAGGCCAGGCCAGTCTCCAAAGCGCCTAATCTTTCTGGTG TCTAAACTGGACTCTGGAGTCCCTGACAGGTTCACTGGCAGTGGATCAGGGACAGAT TTCACACTGAAAATCAGCAGAGTGGAGGCTGAAGATTTGGGAATTTTTTATTGCTGG CAAAATACACATTTTCCGTGGACGTTCGGTGGAGGCACCAAGCTGGAAATCAAACG GGCTGATGCTGCACCAACTGTATCCATCTTCCCACCATCCAGTGAGCAGTTAACATC TGGAGGTGCCTCAGTCGTGTGCTTCTTGAACAACTTCTACCCCAAAGACATCAATGT CAAGTGGAAGATTGATGGCAGTGAACGACAAAATGGCGTCCTGAACAGTTGGACTG ATCAGGACAGCAAAGACAGCACCTACAGCATGAGCAGCACCCTCACGTTGACCAAG GACGAGTATGAACGACATAACAGCTATACCTGTGAGGCCACTCACAAGACATCAAC TTCACCCATTGTCAAGAGCTTCAACAGGAATGAGTGTTAG (SEQ ID NO:1) that encodes the 7F10 light chain including the amino acids

(SEQ ID NO: 2)
MMSPAQFLFLLVLWIRETNGDVVLTQTPLTLSVTIGQPASISCKSSQSL
LDSDGKTYLN¹WLLQRPGQSPKRLIFLVSKLDS²GVPDRFTGSGSGTDFT
LKISRVEAEDLGIFYCWQNTHFPWT³FGGGTKLEIKRADAAPTVSIFPP
SSEQLTSGGASVVCFLNNFYPKDINVKWKIDGSERQNGVLNSWTDQDSK
DSTYSMSSTLTLTKDEYERHNSYTCEATHKTSTSPIVKSFNRNEC
¹ = LCDR1, SEQ ID NO: 27; ² = LCDR2, SEQ ID NO: 28; ³ = LCDR3, SEQ ID NO: 29.

A polynucleotide encoding the 7F10 heavy chain includes the nucleic acid sequence ATGTCCAATGTCCTCTCCTCAGACACTGAACACACTGACTCTAACCATGGGATGGAA CTGGATCTTTCTCTTTCTCCTGTCAGGAACTGCAGGTGTCCTCTCTGAGGTCCGGCTG CAACAGTCTGGACCTGAGCTGGTGAAGCCTGGGGCTTCAGTGAAGATATCCTGCAA GACTTCTGGATACACATTCACTGAATACACCATGCACTGGGTGAAGCAGAGCCATG GAAAGAGCCTTGAGTGGATTGGAGGTATTCATCCTAACAATGGTGGTACTAACTACA AGCAGAAGTTTAAGGGCAGGGCCACATTGACTGTCGACAAGTCCTCCAGCACAGCC TACATGGAGCTCCGCGGCCTGACATCTGAGGATTCTGCAGTCTATTACTGTGCACTG GCGACGGGGTTTGCTTACTGGGGCCAAGGGACTCTGGTCACTGTCTCTGCAGCCAAA ACAACACCCCCATCAGTCTATCCACTGGCCCCTGGGTGTGGAGATACAACTGGTTCC TCCGTGACTCTGGGATGCCTGGTCAAGGGCTACTTCCCTGAGTCAGTGACTGTGACC TGGAACTCT (SEQ ID NO:3) that encodes the 7F10 heavy chain including the amino acids

(SEQ ID NO: 4)
MSSPQTLNTLTLTMGWNWIFLFLLSGTAGVLSEVRLQQSGPELVKPGAS
VKISCKTSGYTFTEYTMH¹WVKQSHGKSLEWIGGIHPNNGGTNYKQKFK
G²RATLTVDKSSSTAYMELRGLTSEDSAVYYCALATGFAY³WGQGTLVTV
SAAKTTPPSVYPLAPGCGDTTGSSVTLGCLVKGYFPESVTVTWNS
¹ = HCDR1, SEQ ID NO: 30; ² = HCDR2, SEQ ID NO: 31; ³ = HCDR3, SEQ ID NO: 32.

A polynucleotide encoding the 3A12 light chain includes the nucleic acid sequence ATGAAGTTGCCTGTTAGGCTGTTGGTGCTGATGTTCTGGATTCCTGCTTCCAGCAGTG ATGTTGTGATGACCCAAATTCCACTCTCCCTGCCTGTCAGTCTTGGAGATCAAGCCTC CATCTCTTGCAGATCTAGTCAGAGCCTTGTACACAGTAATGGAAACACCTATTTACA TTGGTACCTGCAGAAGCCAGGCCAGTCTCTAAAGCTCCTGATCTACAAAGTTTCCAA CCGATTTTCTGGGGTCCCAGACAGGTTCAGTGGCAGTGGATCAGGGACAGATTTCAC ACTCAAGATCAGCAGAGTGGAGGCTGAGGATCTGGGAGTTTATTTCTGCTCTCAAAG TACACATGTTCCGTGGACGTTCGGTGGAGGCACCAAGCTGGAAATCAAACGGGCTG ATGCTGCACCAACTGTATCCATCTTCCCACCATCCAGTGAGCAGTTAACATCTGGAG GTGCCTCAGTCGTGTGCTTCTTGAACAACTTCTACCCCAAAGACATCAATGTCAAGT GGAAGATTGATGGCAGTGAACGACAAAATGGCGTCCTGAACAGTTGGACTGATCAG GACAGCAAAGACAGCACCTACAGCATGAGCAGCACCCTCACGTTGACCAAGGACGA GTATGAACGACATAACAGCTATACCTGTGAGGCCACTCACAAGACATCAACTTCAC CCATTGTCAAGAGCTTCAACAGGAATGAGTGTTAG (SEQ ID NO:5) that encodes the 3A12 chain acids light including the amino

(SEQ ID NO: 6)
MKLPVRLLVLMFWIPASSSDVVMTQIPLSLPVSLGDQASISCRSSQSLV
HSNGNTYLH¹WYLQKPGQSLKLLIYKVSNRFS²GVPDRFSGSGSGTDFTL
KISRVEAEDLGVYFCSQSTHVPWT³FGGGTKLEIKRADAAPTVSIFPPS
SEQLTSGGASVVCFLNNFYPKDINVKWKIDGSERQNGVLNSWTDQDSKD
STYSMSSTLTLTKDEYERHNSYTCEATHKTSTSPIVKSFNRNEC
¹ = LCDR1, SEQ ID NO: 33; ² = LCDR2, SEQ ID NO: 34; ³ = LCDR3, SEQ ID NO: 35.

A polynucleotide encoding the 3A12 heavy chain includes the nucleic acid sequence ATGAAATGCAGCTGGGTTATCTTCTTCCTGATGGCAGTGGTTACAGGGGTCAATTCA GAGGTTCAGCTGCAGCAGTCTGGGGCAGAGCTTGTGAAGCCAGGGGCCTCAGTCAA GTTGTCCTGCACAGTTTCTGGCCTCAACATTAAAGACACCTACATACACTGGGTGAA ACAGAGGCCTGAACAGGGCCTGGAGTGGATTGGAAACATTGATCCTGCGAATGGTG ATACTAAATATGACCCGAAGTTCCAGGGCAAGGCCACTGTAACAACAGTCACATCC TCCAACACAGCCTACCTACAACTCAGCAGCCTGACATCTGAGGACACTGCCGTCTAT TACTGTGCCCTTCCCATGCTTTACTGGGGTCAAGGAACCTCAGTCACCGTCTCCTCA GCCAAAACGACACCCCCATCTGTCTATCCACTGGCCCCTGGATCTGCTGCCCAAACT AACTCCATGGTGACCCTGGGATGCCTGGTCAAGGGCTATTTCCCTGAGCCAGTGACA GTGACCTGGAACTCT (SEQ ID NO:7) that encode the 3A12 heavy chain including amino acids

(SEQ ID NO: 8)
MKCSWVIFFLMAVVTGVNSEVQLQQSGAELVKPGASVKLSCTVSGLNIK
DTYIH¹WVKQRPEQGLEWIGNIDPANGDTKYDPKFQG²KATVTTVTSSNT
AYLQLSSLTSEDTAVYYCALPMLY³WGQGTSVTVSSAKTTPPSVYPLAP
GSAAQTNSMVTLGCLVKGYFPEPVTVTWNS
¹ = HCDR1, SEQ ID NO: 36; ² = HCDR2, SEQ ID NO: 37; ³ = HCDR3, SEQ ID NO: 38.

A polynucleotide encoding the 5B8 light chain includes the nucleic acid sequence ATGCATTTTCAAGTGCAGATTTTCAGCTTCCTGCTAATCAGTGCCTCAGTCATAATGT CCAGAGGACAAATTGTTCTCTCCCAGTCTCCAGCAATCATGTCTGCATCTCCAGGGG AGAAGGTCACCATAACCTGCAGTGCCAGCTCAAGTGTGAGTTACATGCACTGGTTCC AGCAGAAGCCAGGCACTTCTCCCAAACTCTGGATTTATAGCACATCCAACCTGGCTT CTGGCGTCCCTGCTCGCTTCAGTGGCAGTGGATCTGGGACCTCTTACTCTCTCACAAT CAGCCGAATGGAGGCTGAAGATGCTGCCACTTATTACTGCCAGCAAAGGAGTAGTT ACCCCCTCACGTTCGGTGCTGGGACCAAGCTGGAGCTGAAACGGGCTGATGCTGCA CCAACTGTATCCATCTTCCCACCATCCAGTGAGCAGTTAACATCTGGAGGTGCCTCA GTCGTGTGCTTCTTGAACAACTTCTACCCCAAAGACATCAATGTCAAGTGGAAGATT GATGGCAGTGAACGACAAAATGGCGTCCTGAACAGTTGGACTGATCAGGACAGCAA AGACAGCACCTACAGCATGAGCAGCACCCTCACGTTGACCAAGGACGAGTATGAAC GACATAACAGCTATACCTGTGAGGCCACTCACAAGACATCAACTTCACCCATTGTCA AGAGCTTCAACAGGAATGAGTGTTAG (SEQ ID NO:9) that encodes a 5B8 light chain including the amino acids

(SEQ ID NO: 10)
MHFQVQIFSFLLISASVIMSRGQIVLSQSPAIMSASPGEKVTITCSASS
SVSYMH¹WFQQKPGTSPKLWIYSTSNLAS²GVPARFSGSGSGTSYSLTIS
RMEAEDAATYYCQQRSSYPLT³FGAGTKLELKRADAAPTVSIFPPSSEQ
LTSGGASVVCFLNNFYPKDINVKWKIDGSERQNGVLNSWTDQDSKDSTY
SMSSTLTLTKDEYERHNSYTCEATHKTSTSPIVKSFNRNEC
¹ = CDR1, SEQ ID NO: 39; ² = CDR2, SEQ ID NO: 40; ³ = CDR3, SEQ ID NO: 41.

A polynucleotide encoding the 5B8 heavy chain includes the nucleic acid sequence ATGAGCACTGAACACGGACCCCTCACCATGAACTTCGGGCTCAGCTTGATTTTCCTT GTCCTTGTTTTAAAAGGTGTCCAGTGTGAAGTGCAGCTGGTGGAGTCTGGGGGAGGC TTAGTGAAGCCTGGAGGATCCCTGAAACTCTCCTGTGCAGCCTCTGGATTCAGTTTC AGAGACTATCTCATGTATTGGGTTCGCCAGACCCCGGAAAAGAGGCTGGAGTGGGT CGCAATCATTAGAGACGGTGGAAGTTACACCTACTATCCAGACAGTGTGAAGGGGC GGTTCACCATCTCCAGCGACAACGCCGAGAAAAACCTTTACCTGCAAATGAGCAGT CTGAAGTCTGAGGACACAGCCATGTATTACTGTGCAAGAGAGAAGATGATTACGAC AGGGTTTGTTATGGACTACTGGGGTCAAGGAACCTCAGTCACCGTCTCCTCAGCCAA AACAACAGCCCCATCGGTC (SEQ ID NO:11) that encodes a 5B8 heavy chain including the amino acids

(SEQ ID NO: 12)
MSTEHGPLTMNFGLSLIFLVLVLKGVQCEVQLVESGGGLVKPGGSLKLS
CAASGFSFRDYLMY¹WVRQTPEKRLEWVAIIRDGGSYTYYPDSVKG²RFT
ISSDNAEKNLYLQMSSLKSEDTAMYYCAREKMITTGFVMDY³WGQGTSV
TVSSAKTTAPSV
¹ = CDR1, SEQ ID NO: 42; ² = CDR2, SEQ ID NO: 43; ³ = CDR3, SEQ ID NO: 44.

Mouse hybridoma cell lines 8E8, 6E7, and 21B8 produce antibodies against P1.

A polynucleotide encoding the 8E8 light chain includes the nucleic acid sequence ATGAAGTTGCCTGTTAGGCTGTTGGTGCTGATGTTCTGGATTCCTGCTTCCAGCAGTG ATGTTGTGATGACCCAAACTCCACTCTCCCTGCCTGTCAGTCTTGGAGATCAAGCCT CCATCTCTTGCAGATCTAGTCAGAGCCTTGTACACAGTAATGGAAACACCTATTTAC ATTGGTACCTGCAGAAGCCAGGCCAGTCTCCAAAGCTCCTGATCTCCAAAGTTTCCA ACCGATTTTCTGGGGTCCCAGACAGGTTCAGTGGCAGTGGATCAGGGACAGATTTCA CACTCAAGATCAGCAGAGTGGAGGCTGAGGATCTGGGGATTTATTTCTGCTCTCAAA GTACACATTTTCCGTGGACGTTCGGTGGAGGCACCAAGCTGGAAATCAAACGGGCT GATGCTGCACCAACTGTATCCATCTTCCCACCATCCAGTGAGCAGTTAACATCTGGA GGTGCCTCAGTCGTGTGCTTCTTGAACAACTTCTACCCCAAAGACATCAATGTCAAG TGGAAGATTGATGGCAGTGAACGACAAAATGGCGTCCTGAACAGTTGGACTGATCA GGACAGCAAAGACAGCACCTACAGCATGAGCAGCACCCTCACGTTGACCAAGGACG AGTATGAACGACATAACAGCTATACCTGTGAGGCCACTCACAAGACATCAACTTCA CCCATTGTCAAGAGCTTCAACAGGAATGAGTGTTAG (SEQ ID NO:13) that encodes a 8E8 light chain including the amino acids

(SEQ ID NO: 14)
MKLPVRLLVLMFWIPASSSDVVMTQTPLSLPVSLGDQASISCRSSQSLV
HSNGNTYLH¹WYLQKPGQSPKLLISKVSNRFS²GVPDRFSGSGSGTDFTL
KISRVEAEDLGIYFCSQSTHFPWT³FGGGTKLEIKRADAAPTVSIFPPS
SEQLTSGGASVVCFLNNFYPKDINVKWKIDGSERQNGVLNSWTDQDSKD
STYSMSSTLTLTKDEYERHNSYTCEATHKTSTSPIVKSFNRNEC
¹ = CDR1, SEQ ID NO: 45; ² = CDR2, SEQ ID NO: 46; ³ = CDR3, SEQ ID NO: 47.

A polynucleotide encoding the 8E8 heavy chain includes the nucleic acid sequence ATGGACTGGACCTGGGTCTTTCTCTTCCTCCTGTCAGTAACTGCAGGTGTCCACTCCC AGGTTCAGCTGCAGCAGTCTGGAGCTGAACTGATGAAGCCTGGGGCCTCAGTGAAG ATATCCTGCAAGGCTACTGGCTACTCATTCAGTGCCTACTGGATAGATTGGGTAAAG CAGAGGCCTGGACATGGCCTTGAGTGGGTTGGAGAGATTTTACCTGGAAGTGGTAG TACTTACTACAATGAGAAGTTCAAGGGCAAGGCCACATTCACTGCAGATACATCCTC CAACACAGCCTACATGCAACTCAGCAGCCTGACATCTGAGGACTCTGCCGTCTATTT CTGTGCAATTTCGATGGACTACTGGGGTCAAGGAACCTCAGTCACCGTCTCCTCAGC CAAAACAACAGCCCCATCGGTCTATCCACTGGCCCCTGTGTGTGGAGATACAACTGG CTCCTCGGTGACTCTAGGATGCCTGGTCAAGGGTTATTTCCCTGAGCCAGTGACCTT GACCTGGAACTCT (SEQ ID NO:15) that encodes a 8E8 heavy chain including the amino acids

(SEQ ID NO: 16)
MDWTWVFLFLLSVTAGVHSQVQLQQSGAELMKPGASVKISCKATGYSFS
AYWID¹WVKQRPGHGLEWVGEILPGSGSTYYNEKFKG²KATFTADTSSNT
AYMQLSSLTSEDSAVYFCAISMDY³WGQGTSVTVSSAKTTAPSVYPLAP
VCGDTTGSSVTLGCLVKGYFPEPVTLTWNS
¹ = CDR1, SEQ ID NO: 48; ² = CDR2, SEQ ID NO: 49; ³ = CDR3, SEQ ID NO: 50.

A polynucleotide encoding the 6E7 light chain includes the nucleic acid sequence ATGAAGTTGCCTGTTAGGCTGTTGGTGCTGATGTTCTGGATTCCTGGTTCCAGCAGTG ATGTTTTGATGACCCAAACTCCACTCTCCCTGCCTGTCAGTCTTGGAAATCAAGCCTC CATCTCTTGCAAATCTAGTCAAAGCATTGTGCATAGTAATGGAAACACCTATTTAAA ATGGTACCTGCTGAAACCAGGCCAGTCTCCAAAGCTCCTGATCTACAAAGTTTCCAA CCAATTTTCTGGGGTCCCAAACAGGTTCAGTGGCAGTGGATCAGGGACAAATTTCAC ACTCAAAATCAGCAAAGTGGAGACTGAGGATCTGGGAGTTTATTACTGCTTTCAAGG TTCACATGTTCCATTCACGTTCGGCTCGGGGACAAAGTTGGAAATAAAACGGGCTGA TGCTGCACCAACTGTATCCATCTTCCCACCATCCAGTGAGCAGTTAACATCTGGAGG TGCCTCAGTCGTGTGCTTCTTGAACAACTTCTACCCCAAAGACATCAATGTCAAGTG GAAGATTGATGGCAGTGAACGACAAAATGGCGTCCTGAACAGTTGGACTGATCAGG ACAGCAAAGACAGCACCTACAGCATGAGCAGCACCCTCACGTTGACCAAGGACGAG TATGAACGACATAACAGCTATACCTGTGAGGCCACTCACAAAACATCAACTTCACCC ATTGTCAAGAGCTTCAACAGGAATGAGTGTTAG (SEQ ID NO:17) that encodes a 6E7 light chain including the amino acids

(SEQ ID NO: 18)
MKLPVRLLVLMFWIPGSSSDVLMTQTPLSLPVSLGNQASISCKSSQSIV
HSNGNTYLK¹WYLLKPGQSPKLLIYKVSNQFS²GVPNRFSGSGSGTNFTL
KISKVETEDLGVYYCFQGSHVPFT³FGSGTKLEIKRADAAPTVSIFPPS
SEQLTSGGASVVCFLNNFYPKDINVKWKIDGSERQNGVLNSWTDQDSKD
STYSMSSTLTLTKDEYERHNSYTCEATHKTSTSPIVKSFNRNEC
¹ = CDR1, SEQ ID NO: 51, ² = CDR2, SEQ ID NO: 52, ³ = CDR3, SEQ ID NO: 53.

A polynucleotide encoding the 6E7 heavy chain includes the nucleic acid sequence ATGAACTTCGGGCTCAGATTGATTTTCCTTGTCCTTGTTTTAAAAGGTGTCCTGTGTG ACGTGAAGCTCGTGGAGTCTGGGGGAGGCTTAGTGAAGCTTGGAGGGTCCCTGAAA CTCTCCTGTGCAGCCTCTGGATTCACTTTCAGTACCTCTTACATGTCTTGGGTTCGCC AGACTCCAGAGAAGAGGCTGGAGTTGGTCGCAGCCATTAATGGTAATGGTGGTGGC ACCTACTATCCAGACACTGTGAAGGGCCGATTCACCATCTCCAGAGACAATGCCAA GAACACCCTGTACCTGCAAATGAGTAGTCTGAAGTCTGAGGACACAGCCTTGTATTA CTGTGTAAGACATGGGGGTAACTCCTACTGGTATTTCGATGTCTGGGGCGCAGGGAC CACGGTCACCGTCTCCTCAGCCAAAACAACAGCCCCATCGGTCTATCCACTGGCCCC TGTGTGTGGAGATACAACTGGCTCCTCGGTGACTCTAGGATGCCTGGTCAAGGGTTA TTTCCCTGAGCCAGTGACCTTGACCTGGAACTCT (SEQ ID NO:19) that encodes a 6E7 heavy chain including the amino acids

(SEQ ID NO: 20)
MNFGLRLIFLVLVLKGVLCDVKLVESGGGLVKLGGSLKLSCAASGFTFS
TSYMS¹WVRQTPEKRLELVAAINGNGGGTYYPDTVKG²RFTISRDNAKNT
LYLQMSSLKSEDTALYYCVRHGGNSYWYFDV³WGAGTTVTVSSAKTTAP
SVYPLAPVCGDTTGSSVTLGCLVKGYFPEPVTLTWNS
¹ = CDR1, SEQ ID NO: 54, ² = CDR2, SEQ ID NO: 55, ³ = CDR3, SEQ ID NO: 56.

A polynucleotide encoding the 21B8 light chain includes the nucleic acid sequence ATGGGCATCAAGATGGAGACACATTCTCAGGTCTTTGTATACATGTTGCTGTGGTTG TCTGGTGTTGAAGGAGACATTGTGATGACCCAGTCTCACAAATTCCTGTCCACATCA GTAGGAGACAGGGTCAGCATCACCTGCAAGGCCAGTCAGGATGTGGGTACTGCTAT AGCCTGGTTTCAACAGAGACCAGGGCAATCTCCTAAACTACTGATTTACTGGGCATC CACCCGGCACACTGGAGTCCCTGATCGCTTCACAGGCATTGGATCTGGGACAGATTT CACTCTCACCATTAGCAATGTGCAGTCTGAAGACTTGGCAGATTATTTCTGTCAGCA ATATACCAGCTATCCGTGGACGTTCGGTGGAGGCACCAAGCTGGAAATCAAACGGG CTGATGCTGCACCAACTGTATCCATCTTCCCACCATCCAGTGAGCAGTTAACATCTG GAGGTGCCTCAGTCGTGTGCTTCTTGAACAACTTCTACCCCAAAGACATCAATGTCA AGTGGAAGATTGATGGCAGTGAACGACAAAATGGCGTCCTGAACAGTTGGACTGAT CAGGACAGCAAAGACAGCACCTACAGCATGAGCAGCACCCTCACGTTGACCAAGGA CGAGTATGAACGACATAACAGCTATACCTGTGAGGCCACTCACAAGACATCAACTT CACCCATTGTCAAGAGCTTCAACAGGAATGAGTGTTAG (SEQ ID NO:21) that encodes a 21B8 light chain including the amino acids

(SEQ ID NO: 22)
MGIKMETHSQVFVYMLLWLSGVEGDIVMTQSHKFLSTSVGDRVSITCKA
SQDVGTAIA¹WFQQRPGQSPKLLIYWASTRHT²GVPDRFTGIGSGTDFTL
TISNVQSEDLADYFCQQYTSYPWT³FGGGTKLEIKRADAAPTVSIFPPS
SEQLTSGGASVVCFLNNFYPKDINVKWKIDGSERQNGVLNSWTDQDSKD
STYSMSSTLTLTKDEYERHNSYTCEATHKTSTSPIVKSFNRNE
¹ = CDR1, SEQ ID NO: 57; ² = CDR2, SEQ ID NO: 58; ³ = CDR3, SEQ ID NO: 59.

A polynucleotide encoding the 21B8 heavy chain includes the nucleic acid sequence ATGGAATGGCCTTGTATCTTTCTCTTCCTCCTGTCAGTAACTGAAGGTGTCCACTCCC AGGTTCAGCTGCAGCAGTCTGGGGCTGAGGTGATGAGGCCTGGGTCCTCAGTGAAG ATTTCCTGCAAGGCTTCTGGCTATGCATTCAGTAGTCACTGGATGAACTGGGTGAAG CAGAGGCCTGGACAGGGTCTTGAGTGGATTGGACAGATTTATCCTGGAGATGATTAT AATAAATCCAATGGAAAGTTTCAGGGTAGAGCCACACTGACTGCAGACAAATCCTC CAGCACAGCCTACATGCAGCTCAGCAGCCTAACATCTGAGGACTCTGCGGTCTATTT CTGTGCAACCCCAGTGGGGAACTATGCTATGGACTACTGGGGTCAAGGAACCTCAG TCACCGTCTCCTCAGCCAAAACAACAGCCCCATCGGTCTATCCACTGGCCCCTGTGT GTGGAGATACAACTGGCTCCTCGGTGACTCTAGGATGCCTGGTCAAGGGTTATTTCC CTGAGCCAGTGACCTTGACCTGGAACTCT (SEQ ID NO:23) that encodes a 21B8 heavy chain including the amino acids

(SEQ ID NO: 24)
MEWPCIFLFLLSVTEGVHSQVQLQQSGAEVMRPGSSVKISCKASGYAFS
SHWMN¹WVKQRPGQGLEWIGQIYPGDDYNKSNGKFQG²RATLTADKSSST
AYMQLSSLTSEDSAVYFCATPVGNYAMDY³WGQGTSVTVSSAKTTAPSV
YPLAPVCGDTTGSSVTLGCLVKGYFPEPVTLTWNS
¹ = CDR1, SEQ ID NO: 60; ² = CDR2, SEQ ID NO: 61; ³ = CDR3, SEQ ID NO: 62.

Nucleotide sequences encoding the complete variable regions of each antibody are provided as SEQ ID NOs: 1, 3, 5, 7, 9, 11, 13, 15, 17, 19, 21, and 23.

Also encompassed are antigen-binding fragments (e.g., Fab, Fab′, F(ab′)2, Fv, scFv), chimeric antibodies, humanized antibodies, and other derivatives that retain the six CDRs listed above or that compete with the referenced antibodies for binding to CARDS toxin or P1.

Monoclonal antibodies to the polypeptides and/or fragments of this invention are prepared using any technique, which provides for the production of antibody molecules by continuous cell lines in culture. These include, but are not limited to, the hybridoma technique, the human B-cell hybridoma technique, and the EBV-hybridoma technique (Kohler et al. 1975, Nature 256:495-497; Kozboretal, 1985, J. Immunol.)

### II. Polypeptide Compositions

Modifications and/or changes may be made in the amino acid composition of polypeptides, and thus the present invention contemplates variation in sequences of the polypeptides, and nucleic acids coding therefor, where they are nonetheless able retain substantial activity with respect to the therapeutic, preventative, and curative aspects of the present invention.

A biological functional equivalent may comprise a polynucleotide that has been engineered to contain distinct sequences while at the same time retaining the capacity to encode the “parent” polypeptide. This can be accomplished through the degeneracy of the genetic code. In one example, one of skill in the art may wish to introduce a restriction enzyme recognition sequence into a polynucleotide while not disturbing the ability of that polynucleotide to encode a protein.

In another example, a polynucleotide may encode a biological functional equivalent with more significant changes. Certain amino acids may be substituted for other amino acids in a protein structure without appreciable loss of interactive binding capacity with structures such as, for example, antigen-binding regions of antibodies, binding sites on substrate molecules, receptors, and such like. So-called “conservative” changes do not disrupt the biological activity of the protein, as the structural change is not one that impinges on the protein's ability to carry out its designed function. It is thus contemplated by the inventors that various changes may be made in the sequence of genes and proteins disclosed herein, while still fulfilling the goals of the present invention.

In terms of functional equivalents, it is well understood by the skilled artisan that, inherent in the definition of a “biologically functional equivalent” protein and/or polynucleotide, is the concept that there is a limit to the number of changes that may be made within a defined portion of the molecule while retaining a molecule with an acceptable level of equivalent biological activity. Biologically functional equivalents are thus defined herein as those proteins (and polynucleotides) in selected amino acids (or nucleotides) may be substituted. In certain aspects, a polypeptide is 80, 85, 90, 92, 94, 96, 98, or 100% identical to the wildtype form of the polypeptide. In certain aspects, polypeptide(s) 80, 85, 90, 92, 94, 96, 98, or 100% identical to SEQ ID NO:2, 4, 6, 8, 10, 12, 14, 16, 18, 20, 22, or 24 are used or nucleic acids encoding the same.

In general, the shorter the length of the molecule, the fewer changes that can be made within the molecule while retaining function. Longer domains may have an intermediate number of changes. The full-length protein will have the most tolerance for a larger number of changes. However, it must be appreciated that certain molecules or domains that are highly dependent upon their structure may tolerate little or no modification. Function of a polypeptide can be determined by using various assays know to detect the activity of the polypeptide of interest.

Amino acid substitutions are generally based on the relative similarity of the amino acid side-chain substituents, for example, their hydrophobicity, hydrophilicity, charge, size, and/or the like. An analysis of the size, shape and/or type of the amino acid side-chain substituents reveals that arginine, lysine, and/or histidine are all positively charged residues; that alanine, glycine, and/or serine are all a similar size; and/or that phenylalanine, tryptophan, and/or tyrosine all have a generally similar shape. Therefore, based upon these considerations, arginine, lysine, and/or histidine; alanine, glycine, and/or serine; and/or phenylalanine, tryptophan, and/or tyrosine are defined herein as biologically functional equivalents.

To effect more quantitative changes, the hydropathic index of amino acids may be considered. Each amino acid has been assigned a hydropathic index on the basis of their hydrophobicity and/or charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and/or arginine (−4.5).

The importance of the hydropathic amino acid index in conferring interactive biological function on a protein is generally understood in the art (Kyte & Doolittle, 1982). It is known that certain amino acids may be substituted for other amino acids having a similar hydropathic index and/or score and/or still retain a similar biological activity. In making changes based upon the hydropathic index, the substitution of amino acids whose hydropathic indices are within ±2 is preferred, those that are within ±1 are particularly preferred, and/or those within ±0.5 are even more particularly preferred.

It also is understood that the substitution of like amino acids can be made effectively on the basis of hydrophilicity. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within ±2 is preferred, those that are within +1 are particularly preferred, and/or those within ±0.5 are even more particularly preferred.

The antibodies or functional fragments thereof (antibody fragments) of the disclosed subject matter can be generated from any species. The antibodies or functional fragments thereof described herein can be labeled or otherwise conjugated to various chemical or biomolecule moieties, for example, for therapeutic or diagnostic or detection or treatment applications. The moieties can be cytotoxic, for example, bacterial toxins, viral toxins, radioisotopes, and the like. The moieties can be detectable labels, for example, fluorescent labels, radiolabels, biotin, and the like, which are known in the art.

In certain aspects, when the antibodies or peptides are used, e.g., for flow cytometric detection, for scanning laser cytometric detection, or for fluorescent immunoassay, they can usefully be labeled with fluorophores. There are a wide variety of fluorophore labels that can usefully be attached to the antibodies or peptides of the present invention. For flow cytometric applications, both for extracellular detection and for intracellular detection, common useful fluorophores can be fluorescein isothiocyanate (FITC), allophycocyanin (APC), R-phycoerythrin (PE), peridinin chlorophyll protein (PerCP), Texas Red, Cy3, Cy5, fluorescence resonance energy tandem fluorophores such as PerCPCy5.5, PE-Cy5, PE-Cy5.5, PE-Cy7, PE-Texas Red, and APC-Cy7. Other fluorophores include, inter alia, Alexa Fluor® 350, Alexa Fluor® 488, Alexa 25 Fluor® 532, Alexa Fluor® 546, Alexa Fluor® 568, Alexa Fluor® 594, Alexa Fluor® 647 (monoclonal antibody labeling kits available from Molecular Probes, Inc., Eugene, OR, USA), BODIPY dyes, such as BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, Texas Red (available from Molecular Probes, Inc., Eugene, OR, USA), and Cy2, Cy3, Cy3.5, Cy5, Cy5.5, Cy7, all of which are also useful for fluorescently labeling the antibodies of the present invention. For secondary detection using labeled avidin, streptavidin, captavidin or neutravidin, the antibodies of the present invention can usefully be labeled with biotin. When the antibodies of the present invention are used, e.g., for western blotting applications, they can usefully be labeled with radioisotopes, such as 33P, 32P, 35S, 3H, and 1251. As another example, when the antibodies of the present invention are used for radioimmunotherapy, the label can usefully be 3H, 228Th, 227Ac, 225Ac, 223Ra, 213Bi, 212Pb, 212Bi, 211At, 203Pb, 1940s, 188Re, 186Re, 153Sm, 149Tb, 1311, 1251, 111 In, 105Rh, 99mTc, 97Ru, 90Y, 90Sr, 88Y, 72Se, 67Cu, or 47Sc.

### III. Nucleic Acids, Vectors, and Host Cells

In certain embodiments isolated nucleic acid molecules encoding the heavy-chain variable regions, light-chain variable regions, or both, of the disclosed antibodies are provided. Recombinant expression vectors comprising these nucleic acids operably linked to suitable regulatory elements (promoters, enhancers, termination signals, etc.) are also provided, as are host cells (e.g., CHO, HEK293, NS0, PER.C6, or hybridoma cells) transformed or transfected with such vectors.

In certain embodiments the disclosure also provides recombinant expression vectors comprising any of the foregoing nucleic acid molecules operably linked to appropriate transcriptional and translational control sequences. Such control sequences include promoters (e.g., CMV, EF-1α, SV40, RSV, β-actin, or inducible promoters such as tetracycline-regulated systems), enhancers, polyadenylation signals, splice signals, selectable markers (e.g., DHFR, neomycin, puromycin, zeocin), and origins of replication suitable for the intended host. Suitable vector backbones include plasmids (e.g., pcDNA, pCI, pTT, pOptiVEC), viral vectors (lentiviral, adenoviral, AAV), baculovirus vectors, and artificial chromosomes.

Host cells transformed, transfected, or infected with the expression vectors described above are also provided. Representative host cells include, without limitation, Chinese hamster ovary (CHO) cells, HEK293 cells and derivatives thereof, NS0 cells, Sp2/0 cells, PER.C6 cells, BHK cells, hybridoma cells, Escherichia coli, Pichia pastoris, Saccharomyces cerevisiae, and insect cells (e.g., Sf9, High Five). The host cells may be transiently or stably transfected and may be cultured in serum-containing, serum-free, or chemically defined medium as appropriate. Methods for introducing the vectors into host cells (e.g., electroporation, lipofection, calcium phosphate precipitation, viral transduction) and for selecting and amplifying stably transfected cells are well known in the art.

The resulting host cells are capable of producing the disclosed antibodies or antigen-binding fragments thereof in commercially relevant quantities and are useful for large-scale manufacturing under GMP conditions.

### IV. Pharmaceutical Compositions

In light of the current specification, the determination of an appropriate treatment regimen (e.g., dosage, frequency of administration, systemic vs. local, etc.) is within the skill of the art. For administration, the components described herein will be formulated in a unit dosage form (solution, suspension, emulsion, etc.) in association with a pharmaceutically acceptable carrier. Such vehicles are usually nontoxic and non-therapeutic. Examples of such vehicles are water, saline, Ringer's solution, dextrose solution, and Hank's solution. Non-aqueous vehicles such as fixed oils and ethyl oleate may also be used. A preferred vehicle is 5% (w/w) human albumin in saline. The vehicle may contain minor amounts of additives, such as substances that enhance isotonicity and chemical stability, e.g., buffers and preservatives.

The therapeutic compositions described herein, as well as their biological equivalents, can be administered independently or in combination by any suitable route. Examples of parenteral administration include intravenous, intraarterial, intramuscular, intraperitoneal, and the like. The routes of administration described herein are merely an example and in no way limiting.

The dose of the therapeutic compositions administered to an animal, particularly in a human, in accordance with embodiments of the invention, should be sufficient to result in a desired response in the subject over a reasonable time frame. It is known that the dosage of therapeutic compositions depends upon a variety of factors, including the strength of the particular therapeutic composition employed, the age, species, condition or disease state, and the body weight of the animal.

Moreover, dose and dosage regimen, will depend mainly on the type of biological damage to the host, the type of subject, the history of the subject, and the type of therapeutic composition being administered. The size of the dose will be determined by the route, timing and frequency of administration as well as the existence, nature and extent of any adverse side effects that might accompany the administration of a particular therapeutic composition and the desired physiological effect. It is also known that various conditions or disease states, in particular, chronic conditions or disease states, may require prolonged treatment involving multiple administrations.

Therefore, the amount of the therapeutic composition must be effective to achieve an enhanced therapeutic index. If multiple doses are employed, the frequency of administration will depend, for example, on the type of subject. One skilled in the art can ascertain upon routine experimentation the appropriate route and frequency of administration in a given subject that are most effective in any particular case. Suitable doses and dosage regimens can be determined by conventionally known range-finding techniques. Generally, treatment is initiated with smaller dosages, which are less than the optimal dose of the compound. Thereafter, the dosage is increased by small increments until the optimal effect under the circumstances is obtained.

The therapeutic compositions for use in embodiments of the invention generally include carriers. These carriers may be any of those conventionally used and are limited only by the route of administration and chemical and physical considerations, such as solubility and reactivity with the therapeutic agent. In addition, the therapeutic composition may be formulated as polymeric compositions, inclusion complexes, such as cyclodextrin inclusion complexes, liposomes, microspheres, microcapsules, and the like, without limitation.

The pharmaceutically acceptable excipients described herein, for example, vehicles, adjuvants, carriers, or diluents, are well known and readily available. It is preferred that the pharmaceutically acceptable carrier be one which is chemically inert with respect to the therapeutic composition and one that has no detrimental side effects or toxicity under the conditions of use.

The choice of excipient will be determined, in part, by the particular therapeutic composition, as well as by the particular method used to administer the composition. Accordingly, there are a wide variety of suitable formulations of the pharmaceutical composition used in the embodiments of the invention. For example, the non-limiting formulations can be injectable formulations such as, but not limited to, those for intravenous, subcutaneous, intramuscular, intraperitoneal injection, and the like, and oral formulations such as, but not limited to, liquid solutions, including suspensions and emulsions, capsules, sachets, tablets, lozenges, and the like. Non-limiting formulations suitable for parenteral administration include aqueous and non-aqueous isotonic sterile injection solutions, including non-active ingredients such as antioxidants, buffers, bacteriostats, solubilizers, thickening agents, stabilizers, preservatives, surfactants, and the like. The solutions can include oils, fatty acids, including detergents and the like, as well as other well known and common ingredients in such compositions, without limitation.

### V. Therapeutic Methods

The monoclonal antibodies and antigen-binding fragments described herein are useful for the treatment or prevention of Mycoplasma pneumoniae infection and its associated diseases in human and animal subjects. These diseases include, but are not limited to, community-acquired pneumonia, tracheobronchitis, bronchiolitis, pharyngitis, asthma exacerbation, chronic obstructive pulmonary disease exacerbation, and extrapulmonary manifestations such as encephalitis, myocarditis, hemolytic anemia, and reactive arthritis. Certain antibodies (e.g., 7F10, 3A12, and 5B8) specifically bind and neutralize the ADP-ribosyltransferase and vacuolating activities of the CARDS toxin, thereby reducing cytokine storm, ciliostasis, and epithelial cell damage caused by M. pneumoniae. Other antibodies (e.g., 8E8, 6E7, and 21B8) specifically bind the P1 cytadhesin and inhibit bacterial attachment to respiratory epithelium, preventing colonization and subsequent tissue invasion.

An effective amount of one or more of the disclosed antibodies or antigen-binding fragments is administered to a subject diagnosed with, or at risk of developing, M. pneumoniae infection. The antibodies may be administered intravenously, intramuscularly, subcutaneously, intranasally, by inhalation (e.g., nebulized or dry-powder formulation), or by any other route that achieves sufficient concentration at the site of infection. Administration may occur as a single dose or as multiple doses over a period of hours to weeks. In severe or hospitalized cases, the antibodies may be used as monotherapy or in combination with conventional antibiotics (macrolides, tetracyclines, or fluoroquinolones), particularly in macrolide-resistant infections. Prophylactic administration is also contemplated in high-risk settings such as institutional outbreaks.

Pharmaceutical compositions for therapeutic use are formulated to be compatible with the intended route of administration and may include stabilizers (e.g., human serum albumin), preservatives, buffering agents, and tonicity modifiers. Humanized or fully human versions of the disclosed antibodies, or low-immunogenicity fragments (e.g., Fab or scFv), are particularly suitable for repeated administration. Dosages are determined by the attending physician based on factors including the severity of infection, patient age, weight, immune status, and pharmacokinetic profile of the selected antibody, with typical ranges anticipated to be 1-50 mg/kg body weight per dose.

These therapeutic methods provide a targeted biologic approach that addresses both the direct pathogenic effects of M. pneumoniae toxins and the organism's adherence mechanisms, offering advantages over traditional antibiotics in speed of action, efficacy against resistant strains, and reduced disruption of the normal microbiome.

### VI Kits

In another aspect, the present invention provides kits for detecting Mycoplasma. In one embodiment, the kit comprises a solid support, such as a chip, a microtiter plate or a bead or resin having a capture reagent attached thereon, wherein the capture reagent binds a Mycoplasma biomarker or polypeptide or peptide. Thus, for example, the kits of the present invention can comprise mass spectrometry probes for SELDI, such as ProteinChip® arrays or immunoassay supports/reagents. In the case of antibody capture reagents, the kit can comprise a solid support with a reactive surface, and a container comprising the antibody capture reagent.

The kit can also comprise a washing solution or instructions for making a washing solution, in which the combination of the antibody reagent and the washing solution allows capture of the biomarker or biomarkers on the solid support for subsequent detection. The kit may include more than type of adsorbent, each present on a different solid support.

In a further embodiment, such a kit can comprise instructions for suitable operational parameters in the form of a label or separate insert. For example, the instructions may inform a consumer about how to collect the sample, how to wash the probe or the particular biomarkers to be detected.

In yet another embodiment, the kit can comprise one or more containers with biomarker samples, to be used as standard(s) for calibration.

1. An isolated antibody or antigen-binding fragment thereof that specifically binds Mycoplasma pneumoniae, wherein the antibody or fragment comprises:
(a) light chain CDR1-3 of SEQ ID NOs: 27-29 and heavy chain CDR1-3 of SEQ ID NOs: 30-32;
(b) light chain CDR1-3 of SEQ ID NOs: 33-35 and heavy chain CDR1-3 of SEQ ID NOs: 36-38;
(c) light chain CDR1-3 of SEQ ID NOs: 39-41 and heavy chain CDR1-3 of SEQ ID NOs: 42-44;
(d) light chain CDR1-3 of SEQ ID NOs: 45-47 and heavy chain CDR1-3 of SEQ ID NOs: 48-50;
(e) light chain CDR1-3 of SEQ ID NOs: 51-53 and heavy chain CDR1-3 of SEQ ID NOs: 54-56; or
(f) light chain CDR1-3 of SEQ ID NOs: 57-59 and heavy chain CDR1-3 of SEQ ID NOs: 60-62.

2. The antibody or antigen-binding fragment of claim 1, comprising a light chain variable region at least 95% identical to any one of SEQ ID NOs: 2, 6, 10, 14, 18, or 22 and a heavy chain variable region at least 95% identical to a corresponding one of SEQ ID NOs: 4, 8, 12, 16, 20, or 24.

3. The antibody or antigen-binding fragment of claim 1, which is selected from the group consisting of 7F10, 3A12, 5B8, 8E8, 6E7, and 21B8, or an antigen-binding fragment thereof.

4. The antibody or antigen-binding fragment of claim 1, wherein the antibody or fragment specifically binds the CARDS toxin of Mycoplasma pneumoniae and neutralizes ADP-ribosyltransferase activity and/or vacuolating activity of the CARDS toxin.

5. The antibody or antigen-binding fragment of claim 1, wherein the antibody or fragment specifically binds the P1 adhesin of Mycoplasma pneumoniae and inhibits adherence of Mycoplasma pneumoniae to respiratory epithelial cells.

6. The antibody or antigen-binding fragment of claim 1, wherein the antigen-binding fragment is a Fab, Fab′, F(ab′)₂, Fv, scFv, diabody, triabody, tetrabody, minibody, or single-domain antibody.

7. The antibody or antigen-binding fragment of claim 1, wherein the antibody is a humanized antibody, a chimeric antibody, or a fully human antibody.

8. An isolated nucleic acid encoding the antibody or antigen-binding fragment of claim 1.

9. A recombinant vector comprising the nucleic acid of claim 8 operably linked to a promoter.

10. A host cell comprising the recombinant vector of claim 9.

11. A pharmaceutical composition comprising the antibody or antigen-binding fragment of claim 1 and a pharmaceutically acceptable carrier, excipient, or diluent.

12. A method of treating or preventing a Mycoplasma pneumoniae infection in a subject, comprising administering to the subject an effective amount of the antibody or antigen-binding fragment of claim 1 or the pharmaceutical composition of claim 11.

13. The method of claim 12, wherein the infection is macrolide-resistant Mycoplasma pneumoniae.

14. The method of claim 12, wherein the antibody or antigen-binding fragment is administered intravenously, intramuscularly, subcutaneously, intranasally, or by inhalation.

15. A method of detecting Mycoplasma pneumoniae in a biological sample, comprising:
(a) contacting the biological sample with the antibody or antigen-binding fragment of claim 1; and
(b) detecting binding of the antibody or antigen-binding fragment to a Mycoplasma pneumoniae antigen in the sample, wherein binding indicates the presence of Mycoplasma pneumoniae.

16. The method of claim 15, wherein the method is performed in a lateral flow immunoassay, ELISA, immunofluorescence assay, or immunoblot assay.

17. A diagnostic kit for detecting Mycoplasma pneumoniae, comprising the antibody or antigen-binding fragment of claim 1 and at least one ancillary reagent.

18. The kit of claim 17, wherein the kit is configured as a point-of-care rapid antigen test.

19. An antibody or antigen-binding fragment thereof that specifically binds an epitope within amino acids 250-273 of SEQ ID NO: 25 or an epitope within amino acids 570-591 of SEQ ID NO: 25.

20. Use of the antibody or antigen-binding fragment of claim 1 in the manufacture of a medicament for the treatment or prevention of Mycoplasma pneumoniae infection.


SEQUENCE LISTING (62 sequences)

SEQ ID NO: 1 - DNA, 720 bp, synthetic construct
  source 1..720: mol_type = other DNA
atgatgagtcctgcccagttcctgtttctgttagtgctctggattcgggaaaccaacggtgatgttgtgctgacccagactccactcactttgtcggttaccattggacaaccagcctccatctcttgcaagtcaagtcagagcctcttagatagtgatggaaagacatatttgaattggttgttacagaggccaggccagtctccaaagcgcctaatctttctggtgtctaaactggactctggagtccctgacaggttcactggcagtggatcagggacagatttcacactgaaaatcagcagagtggaggctgaagatttgggaattttttattgctggcaaaatacacattttccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 2 - protein, 230 aa, synthetic construct
MMSPAQFLFLLVLWIRETNGDVVLTQTPLTLSVTIGQPASISCKSSQSLLDSDGKTYLNWLLQRPGQSPKRLIFLVSKLDSGVPDRFTGSGSGTDFTLKISRVEAEDLGIFYCFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3 - DNA, 577 bp, synthetic construct
  source 1..577: mol_type = other DNA
atgtccaatgtcctctcctcagacactgaacacactgactctaaccatgggatggaactggatctttctctttctcctgtcaggaactgcaggtgtcctctctgaggtccggctgcaacagtctggacctgagctggtgaagcctggggcttcagtgaagatatcctgcaagacttctggatacacattcactgaatacaccatgcactgggtgaagcagagccatggaaagagccttgagtggattggaggtattcatcctaacaatggtggtactaactacaagcagaagtttaagggcagggccacattgactgtcgacaagtcctccagcacagcctacatggagctccgcggcctgacatctgaggattctgcagtctattactgtgcactggcgacggggtttgcttactggggccaagggactctggtcactgtctctgcagccaaaacaacacccccatcagtctatccactggcccctgggtgtggagatacaactggttcctccgtgactctgggatgcctggtcaagggctacttccctgagtcagtgactgtgacctggaactct

SEQ ID NO: 4 - protein, 190 aa, synthetic construct
MSSPQTLNTLTLTMGWNWIFLFLLSGTAGVLSEVRLQQSGPELVKPGASVKISCKTSGYTFTEYTMHWVKQSHGKSLEWIGGIHPNNGGTNYKQKFKGRATLTVDKSSSTAYMELRGLTSEDSAVYYCALATGFAYWGQGTLVTVSAAKTTPPSVYPLAPGCGDTTGSSVTLGCLVKGYFPESVTVTWNS

SEQ ID NO: 5 - DNA, 717 bp, synthetic construct
  source 1..717: mol_type = other DNA
atgaagttgcctgttaggctgttggtgctgatgttctggattcctgcttccagcagtgatgttgtgatgacccaaattccactctccctgcctgtcagtcttggagatcaagcctccatctcttgcagatctagtcagagccttgtacacagtaatggaaacacctatttacattggtacctgcagaagccaggccagtctctaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 6 - protein, 238 aa, synthetic construct
MKLPVRLLVLMFWIPASSSDVVMTQIPLSLPVSLGDQASISCRSSQSLVHSNGNTYLHWYLQKPGQSLKLLIYKVSNRFSGVPDRFSGSGSGTDFTLKISRVEAEDLGVYFCSQSTHVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 7 - DNA, 525 bp, synthetic construct
  source 1..525: mol_type = other DNA
atgaaatgcagctgggttatcttcttcctgatggcagtggttacaggggtcaattcagaggttcagctgcagcagtctggggcagagcttgtgaagccaggggcctcagtcaagttgtcctgcacagtttctggcctcaacattaaagacacctacatacactgggtgaaacagaggcctgaacagggcctggagtggattggaaacattgatcctgcgaatggtgatactaaatatgacccgaagttccagggcaaggccactgtaacaacagtcacatcctccaacacagcctacctacaactcagcagcctgacatctgaggacactgccgtctattactgtgcccttcccatgctttactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactct

SEQ ID NO: 8 - protein, 175 aa, synthetic construct
MKCSWVIFFLMAVVTGVNSEVQLQQSGAELVKPGASVKLSCTVSGLNIKDTYIHWVKQRPEQGLEWIGNIDPANGDTKYDPKFQGKATVTTVTSSNTAYLQLSSLTSEDTAVYYCALPMLYWGQGTSVTVSSAKTTPPSVYPLAPGSAAQTNSMVTLGCLVKGYFPEPVTVTWNS

SEQ ID NO: 9 - DNA, 708 bp, synthetic construct
  source 1..708: mol_type = other DNA
atgcattttcaagtgcagattttcagcttcctgctaatcagtgcctcagtcataatgtccagaggacaaattgttctctcccagtctccagcaatcatgtctgcatctccaggggagaaggtcaccataacctgcagtgccagctcaagtgtgagttacatgcactggttccagcagaagccaggcacttctcccaaactctggatttatagcacatccaacctggcttctggcgtccctgctcgcttcagtggcagtggatctgggacctcttactctctcacaatcagccgaatggaggctgaagatgctgccacttattactgccagcaaaggagtagttaccccctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 10 - protein, 235 aa, synthetic construct
MHFQVQIFSFLLISASVIMSRGQIVLSQSPAIMSASPGEKVTITCSASSSVSYMHWFQQKPGTSPKLWIYSTSNLASGVPARFSGSGSGTSYSLTISRMEAEDAATYYCQQRSSYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 11 - DNA, 471 bp, synthetic construct
  source 1..471: mol_type = other DNA
atgagcactgaacacggacccctcaccatgaacttcgggctcagcttgattttccttgtccttgttttaaaaggtgtccagtgtgaagtgcagctggtggagtctgggggaggcttagtgaagcctggaggatccctgaaactctcctgtgcagcctctggattcagtttcagagactatctcatgtattgggttcgccagaccccggaaaagaggctggagtgggtcgcaatcattagagacggtggaagttacacctactatccagacagtgtgaaggggcggttcaccatctccagcgacaacgccgagaaaaacctttacctgcaaatgagcagtctgaagtctgaggacacagccatgtattactgtgcaagagagaagatgattacgacagggtttgttatggactactggggtcaaggaacctcagtcaccgtctcctcagccaaaacaacagccccatcggtc

SEQ ID NO: 12 - protein, 157 aa, synthetic construct
MSTEHGPLTMNFGLSLIFLVLVLKGVQCEVQLVESGGGLVKPGGSLKLSCAASGFSFRDYLMYWVRQTPEKRLEWVAIIRDGGSYTYYPDSVKGRFTISSDNAEKNLYLQMSSLKSEDTAMYYCAREKMITTGFVMDYWGQGTSVTVSSAKTTAPSV

SEQ ID NO: 13 - DNA, 717 bp, synthetic construct
  source 1..717: mol_type = other DNA
atgaagttgcctgttaggctgttggtgctgatgttctggattcctgcttccagcagtgatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctccaaagtttccaaccgattttctggggtcccagacaggttcagtggcagtggatcagggacagatttcacactcaagatcagcagagtggaggctgaggatctggggatttatttctgctctcaaagtacacattttccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 14 - protein, 238 aa, synthetic construct
MKLPVRLLVLMFWIPASSSDVVMTQTPLSLPVSLGDQASISCRSSQSLVHSNGNTYLHWYLQKPGQSPKLLISKVSNRFSGVPDRFSGSGSGTDFTLKISRVEAEDLGIYFCSQSTHFPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 15 - DNA, 525 bp, synthetic construct
  source 1..525: mol_type = other DNA
atggactggacctgggtctttctcttcctcctgtcagtaactgcaggtgtccactcccaggttcagctgcagcagtctggagctgaactgatgaagcctggggcctcagtgaagatatcctgcaaggctactggctactcattcagtgcctactggatagattgggtaaagcagaggcctggacatggccttgagtgggttggagagattttacctggaagtggtagtacttactacaatgagaagttcaagggcaaggccacattcactgcagatacatcctccaacacagcctacatgcaactcagcagcctgacatctgaggactctgccgtctatttctgtgcaatttcgatggactactggggtcaaggaacctcagtcaccgtctcctcagccaaaacaacagccccatcggtctatccactggcccctgtgtgtggagatacaactggctcctcggtgactctaggatgcctggtcaagggttatttccctgagccagtgaccttgacctggaactct

SEQ ID NO: 16 - protein, 175 aa, synthetic construct
MDWTWVFLFLLSVTAGVHSQVQLQQSGAELMKPGASVKISCKATGYSFSAYWIDWVKQRPGHGLEWVGEILPGSGSTYYNEKFKGKATFTADTSSNTAYMQLSSLTSEDSAVYFCAISMDYWGQGTSVTVSSAKTTAPSVYPLAPVCGDTTGSSVTLGCLVKGYFPEPVTLTWNS

SEQ ID NO: 17 - DNA, 717 bp, synthetic construct
  source 1..717: mol_type = other DNA
atgaagttgcctgttaggctgttggtgctgatgttctggattcctggttccagcagtgatgttttgatgacccaaactccactctccctgcctgtcagtcttggaaatcaagcctccatctcttgcaaatctagtcaaagcattgtgcatagtaatggaaacacctatttaaaatggtacctgctgaaaccaggccagtctccaaagctcctgatctacaaagtttccaaccaattttctggggtcccaaacaggttcagtggcagtggatcagggacaaatttcacactcaaaatcagcaaagtggagactgaggatctgggagtttattactgctttcaaggttcacatgttccattcacgttcggctcggggacaaagttggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaaaacatcaacttcacccattgtcaagagcttcaacaggaatgagtgttag

SEQ ID NO: 18 - protein, 238 aa, synthetic construct
MKLPVRLLVLMFWIPGSSSDVLMTQTPLSLPVSLGNQASISCKSSQSIVHSNGNTYLKWYLLKPGQSPKLLIYKVSNQFSGVPNRFSGSGSGTNFTLKISKVETEDLGVYYCFQGSHVPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 19 - DNA, 546 bp, synthetic construct
  source 1..546: mol_type = other DNA
atgaacttcgggctcagattgattttccttgtccttgttttaaaaggtgtcctgtgtgacgtgaagctcgtggagtctgggggaggcttagtgaagcttggagggtccctgaaactctcctgtgcagcctctggattcactttcagtacctcttacatgtcttgggttcgccagactccagagaagaggctggagttggtcgcagccattaatggtaatggtggtggcacctactatccagacactgtgaagggccgattcaccatctccagagacaatgccaagaacaccctgtacctgcaaatgagtagtctgaagtctgaggacacagccttgtattactgtgtaagacatgggggtaactcctactggtatttcgatgtctggggcgcagggaccacggtcaccgtctcctcagccaaaacaacagccccatcggtctatccactggcccctgtgtgtggagatacaactggctcctcggtgactctaggatgcctggtcaagggttatttccctgagccagtgaccttgacctggaactct

SEQ ID NO: 20 - protein, 182 aa, synthetic construct
MNFGLRLIFLVLVLKGVLCDVKLVESGGGLVKLGGSLKLSCAASGFTFSTSYMSWVRQTPEKRLELVAAINGNGGGTYYPDTVKGRFTISRDNAKNTLYLQMSSLKSEDTALYYCVRHGGNSYWYFDVWGAGTTVTVSSAKTTAPSVYPLAPVCGDTTGSSVTLGCLVKGYFPEPVTLTWNS

SEQ ID NO: 21 - DNA, 717 bp, synthetic construct
  source 1..717: mol_type = other DNA
atgggcatcaagatggagacacattctcaggtctttgtatacatgttgctgtggttgtctggtgttgaaggagacattgtgatgacccagtctcacaaattcctgtccacatcagtaggagacagggtcagcatcacctgcaaggccagtcaggatgtgggtactgctatagcctggtttcaacagagaccagggcaatctcctaaactactgatttactgggcatccacccggcacactggagtccctgatcgcttcacaggcattggatctgggacagatttcactctcaccattagcaatgtgcagtctgaagacttggcagattatttctgtcagcaatataccagctatccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 22 - protein, 237 aa, synthetic construct
MGIKMETHSQVFVYMLLWLSGVEGDIVMTQSHKFLSTSVGDRVSITCKASQDVGTAIAWFQQRPGQSPKLLIYWASTRHTGVPDRFTGIGSGTDFTLTISNVQSEDLADYFCQQYTSYPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNE

SEQ ID NO: 23 - DNA, 540 bp, synthetic construct
  source 1..540: mol_type = other DNA
atggaatggccttgtatctttctcttcctcctgtcagtaactgaaggtgtccactcccaggttcagctgcagcagtctggggctgaggtgatgaggcctgggtcctcagtgaagatttcctgcaaggcttctggctatgcattcagtagtcactggatgaactgggtgaagcagaggcctggacagggtcttgagtggattggacagatttatcctggagatgattataataaatccaatggaaagtttcagggtagagccacactgactgcagacaaatcctccagcacagcctacatgcagctcagcagcctaacatctgaggactctgcggtctatttctgtgcaaccccagtggggaactatgctatggactactggggtcaaggaacctcagtcaccgtctcctcagccaaaacaacagccccatcggtctatccactggcccctgtgtgtggagatacaactggctcctcggtgactctaggatgcctggtcaagggttatttccctgagccagtgaccttgacctggaactct

SEQ ID NO: 24 - protein, 180 aa, synthetic construct
MEWPCIFLFLLSVTEGVHSQVQLQQSGAEVMRPGSSVKISCKASGYAFSSHWMNWVKQRPGQGLEWIGQIYPGDDYNKSNGKFQGRATLTADKSSSTAYMQLSSLTSEDSAVYFCATPVGNYAMDYWGQGTSVTVSSAKTTAPSVYPLAPVCGDTTGSSVTLGCLVKGYFPEPVTLTWNS

SEQ ID NO: 25 - protein, 591 aa, Mycoplasma pneumoniae
MPNPVRFVYRVDLRSPEEIFEHGFSTLGDVRNFFEHILSTNFGRSYFISTSETPTAAIRFFGSWLREYVPEHPRRAYLYEIRADQHFYNARATGENLLDLMRQRQVVFDSGDREMAQMGIRALRTSFAYQREWFTDGPIAAANVRSAWLVDAVPVEPGHAHHPAGRVVETTRINEPEMHNPHYQELQTQANDQPWLPTPGIATPVHLSIPQAASVADVSEGTSASLSFACPDWSPPSSNGENPLDKCIAEKIDNYNLQSLPQYASSVKELEDTPVYLRGIKTQKTFMLQADPQNNNVFLVEVNPKQKSSFPQTIFFWDVYQRICLKDLTGAQISLSLTAFTTQYAGQLKVHLSVSAVNAVNQKWKMTPQDIAITQFRVSSELLGQTENGLFWNTKSGGSQHDLYVCPLKNPPSDLEELQIIVDECTTHAQFVTMRAASTFFVDVQLGWYWRGYYYTPQLSGWSYQMKTPDGQIFYDLKTSKIFFVQDNQNVFFLHNKLNKQTGYSWDWVEWLKHDMNEDKDENFKWYFSRDDLTIPSVEGLNFRHIRCYADNQQLKVIISGSRWGGWYSTYDKVESNVEDKILVKDGFDRF

SEQ ID NO: 26 - protein, 1627 aa, Mycoplasma pneumoniae
MHQTKKTALSKSTWILILTATASLATGLTVVGHFTSTTTTLKRQQFSYTRPDEVALRHTNAINPRLTPWTYRNTSFSSLPLTGENPGAWALVRDNSAKGITAGSGSQQTTYDPTRTEAALTASTTFALRRYDLAGRALYDLDFSKLNPQTPTRDQTGQITFNPFGGFGLSGAAPQQWNEVKNKVPVEVAQDPSNPYRFAVLLVPRSVVYYEQLQRGLGLPQQRTESGQNTSTTGAMFGLKVKNAEADTAKSNEKLQGAEATGSSTTSGSGQSTQRGGSSGDTKVKALKIEVKKKSDSEDNGQLQLEKNDLANAPIKRSEESGQSVQLKADDFGTALSSSGSGGNSNPGSPTPWRPWLATEQIHKDLPKWSASILILYDAPYARNRTAIDRVDHLDPKAMTANYPPSWRTPKWNHHGLWDWKARDVLLQTTGFFNPRRHPEWFDGGQTVADNEKTGFDVDNSENTKQGFQKEADSDKSAPIALPFEAYFANIGNLTWFGQALLVFGGNGHVTKSAHTAPLSIGVFRVRYNATGTSATVTGWPYALLFSGMVNKQTDGLKDLPFNNNRWFEYVPRMAVAGAKFVGRELVLAGTITMGDTATVPRLLYDELESNLNLVAQGQGLLREDLQLFTPYGWANRPDLPIGAWSSSSSSSHNAPYYFHNNPDWQDRPIQNVVDAFIKPWEDKNGKDDAKYIYPYRYSGMWAWQVYNWSNKLTDQPLSADFVNENAYQPNSLFAAILNPELLAALPDKVKYGKENEFAANEYERFNQKLTVAPTQGTNWSHFSPTLSRFSTGFNLVGSVLDQVLDYVPWIGNGYRYGNNHRGVDDITAPQTSAGSSSGISTNTSGSRSFLPTFSNIGVGLKANVQATLGGSQTMITGGSPRRTLDQANLQLWTGAGWRNDKASSGQSDENHTKFTSATGMDQQGQSGTSAGNPDSLKQDNISKSGDSLTTQDGNAIDQQEATNYTNLPPNLTPTADWPNALSFTNKNNAQRAQLFLRGLLGSIPVLVNRSGSDSNKFQATDQKWSYTDLHSDQTKLNLPAYGEVNGLLNPALVETYFGNTRAGGSGSNTTSSPGIGFKIPEQNNDSKATLITPGLAWTPQDVGNLVVSGTTVSFQLGGWLVTFTDFVKPRAGYLGLQLTGLDASDATQRALIWAPRPWAAFRGSWVNRLGRVESVWDLKGVWADQAQSDSQGSTTTATRNALPEHPNALAFQVSVVEASAYKPNTSSGQTQSTNSSPYLHLVKPKKVTQSDKLDDDLKNLLDPNQVRTKLRQSFGTDHSTQPQPQSLKTTTPVFGTSSGNLSSVLSGGGAGGGSSGSGQSGVDLSPVEKVSGWLVGQLPSTSDGNTSSTNNLAPNTNTGNDVVGVGRLSESNAAKMNDDVDGIVRTPLAELLDGEGQTADTGPQSVKFKSPDQIDFNRLFTHPVTDLFDPVTMLVYDQYIPLFIDIPASVNPKMVRLKVLSFDTNEQSLGLRLEFFKPDQDTQPNNNVQVNPNNGDFLPLLTASSQGPQTLFSPFNQWPDYVLPLAITVPIVVIVLSVTLGLAIGIPMHKNKQALKAGFALSNQKVDVLTKAVGSVFKEIINRTGISQAPKRLKQTSAAKPGAPRPPVPPKPGAPKPPVQPPKKPA

SEQ ID NO: 27 - protein, 16 aa, synthetic construct
KSSQSLLDSDGKTYLN

SEQ ID NO: 28 - protein, 7 aa, synthetic construct
LVSKLDS

SEQ ID NO: 29 - protein, 9 aa, synthetic construct
WQNTHFPWT

SEQ ID NO: 30 - protein, 5 aa, synthetic construct
EYTMH

SEQ ID NO: 31 - protein, 17 aa, synthetic construct
GIHPNNGGTNYKQKFKG

SEQ ID NO: 32 - protein, 6 aa, synthetic construct
ATGFAY

SEQ ID NO: 33 - protein, 16 aa, synthetic construct
RSSQSLVHSNGNTYLH

SEQ ID NO: 34 - protein, 7 aa, synthetic construct
KVSNRFS

SEQ ID NO: 35 - protein, 9 aa, synthetic construct
SQSTHVPWT

SEQ ID NO: 36 - protein, 5 aa, synthetic construct
DTYIH

SEQ ID NO: 37 - protein, 17 aa, synthetic construct
NIDPANGDTKYDPKFQG

SEQ ID NO: 38 - protein, 4 aa, synthetic construct
PMLY

SEQ ID NO: 39 - protein, 10 aa, synthetic construct
SASSSVSYMH

SEQ ID NO: 40 - protein, 7 aa, synthetic construct
STSNLAS

SEQ ID NO: 41 - protein, 9 aa, synthetic construct
QQRSSYPLT

SEQ ID NO: 42 - protein, 5 aa, synthetic construct
DYLMY

SEQ ID NO: 43 - protein, 17 aa, synthetic construct
IIRDGGSYTYYPDSVKG

SEQ ID NO: 44 - protein, 12 aa, synthetic construct
EKMITTGFVMDY

SEQ ID NO: 45 - protein, 16 aa, synthetic construct
RSSQSLVHSNGNTYLH

SEQ ID NO: 46 - protein, 7 aa, synthetic construct
KVSNRFS

SEQ ID NO: 47 - protein, 9 aa, synthetic construct
SQSTHFPWT

SEQ ID NO: 48 - protein, 5 aa, synthetic construct
AYWID

SEQ ID NO: 49 - protein, 17 aa, synthetic construct
EILPGSGSTYYNEKFKG

SEQ ID NO: 50 - protein, 4 aa, synthetic construct
SMDY

SEQ ID NO: 51 - protein, 16 aa, synthetic construct
KSSQSIVHSNGNTYLK

SEQ ID NO: 52 - protein, 7 aa, synthetic construct
KVSNQFS

SEQ ID NO: 53 - protein, 9 aa, synthetic construct
FQGSHVPFT

SEQ ID NO: 54 - protein, 5 aa, synthetic construct
TSYMS

SEQ ID NO: 55 - protein, 17 aa, synthetic construct
AINGNGGGTYYPDTVKG

SEQ ID NO: 56 - protein, 11 aa, synthetic construct
HGGNSYWYFDV

SEQ ID NO: 57 - protein, 11 aa, synthetic construct
KASQDVGTAIA

SEQ ID NO: 58 - protein, 7 aa, synthetic construct
WASTRHT

SEQ ID NO: 59 - protein, 9 aa, synthetic construct
QQYTSYPWT

SEQ ID NO: 60 - protein, 5 aa, synthetic construct
SHWMN

SEQ ID NO: 61 - protein, 17 aa, synthetic construct
QIYPGDDYNKSNGKFQG

SEQ ID NO: 62 - protein, 9 aa, synthetic construct
PVGNYAMDY